(19) United States
(12) Patent Application Publication
(10) Pub. No.: US 2012/0197139 A1
(43) Pub. Date: Aug. 2, 2012

(54) AUTO-DIAGNOSTIC BLOOD MANOMETER

(76) Inventor: Byung Hoon Lee, Seoul (KR)

(21) Appl. No.: 13/383,102

(22) PCT Filed: Sep. 2, 2010
(86) PCT No.: PCT/KR2010/005947

§ 371 Date: Jan. 9, 2012

(30) Foreign Application Priority Data
Jan. 29, 2010 (KR) 10-2010-0008290

(51) Int. Cl. A61B 5/0225; A61B 5/025

(52) U.S. Cl. 600/494

(57) ABSTRACT

The present invention relates to a blood manometer displaying the examined person's heart condition, showing the name of a cardiac disease identified, comprising a cuff pressure control portion for adjusting the air pressure of a cuff; a blood pressure measuring portion having a pressure sensor for measuring blood pressure; a pulse measuring portion having a pulse wave sensor for measuring pulse waves; and a central process-and-control portion which processes by computing the data inputted by the blood pressure measuring portion and the pulse measuring portion, and displays in words the results of the blood pressure that has been measured, such as normal blood pressure, hypertension or hypotension, as well as the result of the pulse that has been measured, such as normal pulse, arrhythmia, tachycardia or bradycardia. The central process-and-control portion is also capable of storing and transmitting the data.

As the blood manometer not only measures blood pressure but displays specific information on the examined person's heart condition, it is helpful for ordinary people to manage their health at home and at work place. If the user sees an abnormal condition displayed, he or she may transmit the data swiftly to a remote-placed specialist for precise diagnosis by telemedicine. This will assist greatly in the prevention and treatment of a heart disease.

### TECHNICAL FIELD

The present invention relates to an auto-diagnostic blood manometer that displays information on the heart condition of the person being examined, showing the name of a cardiac disease identified, at the same time when blood pressure is measured.

### BACKGROUND ART

An ordinary blood manometer, which is intended to measure blood pressure at the contraction phase and at the relaxation phase of the heart operation, comprises a combination of a mercury column for measuring pressure and a stethoscope for detecting korotkoff sound. Various kinds of electronic blood manometers for automatic blood pressure measurement have been known recently.

Examples of the known electronic blood manometers are as follows.

The published Korean Utility Model No. 1979-0001708 (publication date 30 Sep. 1979); the published Korean Utility Model No. 1989-0003515 (publication date 27 May 1989); the published Korean Patent Application No. 1990-0009013 (publication date 17 Dec. 1990); the published Korean Patent No. 10-0827816 (publication date 7 May 2008); the published Korean Patent No. 10-0430144 (publication date 3 May 2004); and the published Korean Patent No. 10-0618624 (publication date 5 Sep. 2006)

The published Korean Utility Model No. 1979-0001708 discloses a blood manometer in which, if pressure is exerted to the cuff, power is applied automatically, the signals detected by the blood stream detector are amplified, with noises filtered, then amplified again. The signals of the amplified blood stream sound is shown through a speaker and by means of a light.

The published Korean Utility Model No. 1989-0003515 discloses an automatic blood manometer, in which an air pump that supplies air to the cuff is housed in a sealed cabin in the body of the blood manometer so as to absorb the noise and the oscillation of the pump.

The published Korean Patent No. 1990-0009013 discloses an automatic blood manometer comprising a first pressure means that exerts pressure on a cuff swiftly, a means to detect pulse waves, a means to forecast maximal blood pressure that forecasts maximal blood pressure from the oscillation of the pulse waves detected by said means to detect pulse waves during the exertion of pressure by the first pressure means, a second pressure means that exerts pressure into the cuff at a speed less than the pressure speed by the first pressure means, a measuring means that measures maximal blood pressure during the pressure exerted by the second pressure means, and an automatic release means that releases the second pressure means in order to measure the minimal blood pressure after measuring the maximal blood pressure by said measuring means, thereby automatically measuring the maximal and minimal blood pressure.

The published Korean Patent No. 10-0827816 discloses an apparatus and method for measuring blood pressure comprising a first measuring means and a second measuring means for measuring the variation in the blood pressure of the person being examined, a pressure adjusting portion for adjusting the pressure of the first and the second measuring means, a signal processing portion that converts the information on the variation of the person's blood pressure measured by the first and the second measuring means into the first and the second pulse wave information, and a processor portion that measures the person's blood pressure in the contraction phase and in the relaxation phase by analyzing the information provided by the first and the second pulse wave information, wherein the blood pressure of the person's contraction phase is measured by the second measuring means when the second pulse begins to be detected after the detection of the first pulse wave, the blood pressure of the relaxation phase is the blood pressure at the time when an inflection point is detected after the conversion of the first pulse wave form or the second pulse wave form into wavelets, and the method for the blood pressure measurement is recorded and stored in a computer so that it may be available for reading when necessary.

The published Korean Patent No. 10-0430114 discloses an electronic blood manometer comprising a computer for control operations capable of transmitting and receiving data, a system controlling means that controls the supply and blocking of air, an air controlling means that receives the control signals outputted from the system controlling means and drives a motor by driving a relay in order to raise and lower blood pressure, an emergency control portion for automatic release by making necessary adjustments by the user when an emergency occurs during the operation of the air controlling means, and a power applying means that applies power received from outside, wherein the blood pressure, the number of pulses, the blood pressure distribution graph are measured and stored automatically, and printed when necessary.

The published Korean Patent No. 10-0618624 discloses a blood pressure measuring system, in which the information on the blood pressure that has been measured may be transmitted to a mobile phone for a long distance communication.

### DETAILED DESCRIPTION OF THE INVENTION

### Technical Problem

The prior arts shown above disclose various measurement devices and methods that make it possible to measure blood pressure precisely. However, as these devices display the result of the measurement by numbers, ordinary people, without a professional medical knowledge, may only know from the numbers shown on the device that their blood pressure or the number of their pulses is high or low, but they may not know what implication these numbers have on their physical health.

Therefore, it happens often that a patient with arrhythmia, angina, hypotension, or hypertension without a subjective symptom may be exposed to a dangerous situation suddenly in an absent-minded state.

It is more economical to prevent a disease than to resort to a medical treatment after a person has fallen ill. Besides, prevention of a disease enhances the chances of a person's stable, healthy life. Therefore, people are more and more interested in preventing diseases and are making greater efforts to maintain good physical conditions. As the danger of a cardiac disease comes in an unpredictable manner, it becomes necessary to develop a blood manometer that displays the examined person's heart condition, showing the name of a disease at the same time when the blood pressure is measured. A blood manometer with such a function enables a person to grasp his or her heart condition specifically by measuring his or her blood pressure so as to visit, when necessary, a specialist physician without delay for further diagnosis and necessary medical treatment.

### Technical Solution

In view of the above problem, a blood manometer which displays the name of a disease at the same time when blood pressure is measured has been developed through various studies and researches.

The object of the present invention is to provide a blood manometer that may be used by ordinary people, wherein the state of the pulse waves is measured at the same time when blood pressure is measured, from which the person being examined, or the user of the blood manometer, may know the specific cardiac disease-related condition she or he is placed in by reading the words displayed.

Another object of the present invention is to provide a blood manometer which displays the examined person's heart condition, showing the name of a cardiac disease identified, wherein blood pressure is measured by the combination of an electronic blood pressure measuring means and a pulse wave measuring means, wherein the heart condition of the person examined, with the specific name of a cardiac disease shown, such as normal blood pressure, hypertension, hypotension, normal pulse, arrhythmia, tachycardia, or bradycardia, is displayed in words, and wherein the storage, outputting and transmission of such data to a remote-placed recipient, including a physician for telemedicine, is possible. Besides, the blood manometer in the present invention is of a portable, convenient size for use.

Another object of the present invention is to provide a blood manometer that displays the name of the patient's disease so that the examined person may be able to read the name of her or his disease when the result of the blood pressure measurement is displayed. Therefore, even if the person does not feel any symptom of a disease, he or she may visit a specialist doctor without delay for necessary medical treatment.

The present invention is explained in detail as follows.

The blood manometer in the present invention has a blood pressure measuring portion comprising a cuff for measuring blood pressure, a cuff pressure control portion that adjusts and controls the air pressure in the cuff, and a pressure sensor that measures blood pressure, wherein a pulse wave measuring portion comprising a pulse wave sensor for measuring pulse waves is provided, and wherein a central process-and-control portion is provided that analyzes and processes the data detected and outputted from both the blood pressure measuring portion and the pulse wave measuring portion, displays on a display portion the heart condition of the examined person, showing the name of a cardiac disease identified, in words, such as normal blood pressure, hypertension, hypotension, normal pulse, arrhythmia, tachycardia, or bradycardia, and stores and transmits the data thus obtained.

The cuff pressure control portion is provided with a pump that supplies compressed air, an air supply portion that injects air into the cuff with a predetermined pressure, and an air exhaust portion for exhausting the air from the cuff.

The blood pressure measuring portion is of a common type provided with a pressure sensor and transmits the measured blood pressure data to the central process-and-control portion after the data are amplified and filtered.

The pulse wave measuring portion having a pulse wave sensor comprises an amplifying portion that amplifies the waveform data of the pulse sound measured by the pulse wave sensor, a filtering portion that filters external noises, an A/D conversion portion that converts the analog waveform data into digital data, and a comparison portion that analyzes the type of the pulse wave and identifies it as a certain type of a pulse wave model. The data of the pulse wave thus identified are inputted into the central process-and-control portion.

The central process-and-control portion consists of a set of computer circuits and controls the overall operations of the cuff pressure control portion, the blood pressure measuring portion and the pulse wave measuring portion according to an inputted program, analyzes the measured data inputted from the blood pressure measuring portion and the pulse wave measuring portion, identifies the specific name of a cardiac disease suffered by the person being examined, stores the analyzed data, and outputs the identified data, i.e. the name of a disease, to the display portion and the output portion.

Also provided is a power supply portion that supplies power adjusting the electric currency to suit the operation of each component. However, as this is of a general type of a known art, a detailed explanation is deemed unnecessary.

The component elements in the present invention are known arts on an individual basis in the field of electronics. However, the characteristic features of the present invention lie in the fact that with the combination of the blood pressure measuring portion and the pulse wave measuring portion, the examined person's heart condition is identified, with the name of a cardiac disease identified shown, and displayed in words. The identified disease name is also available via printing, voice, as well as by wireless transmission to a remote-placed recipient.

The measuring of the blood pressure in the present invention is done via an ordinary measuring method, in which the measuring of blood pressure and the measuring of pulse waves are done at the same time. In other words, a cuff is wound around a designated portion of the body of the person being examined, and when an operation switch (not shown in the drawings) is turned on, power is supplied from the power supply portion to activate all the component portions. This drives the pump with the operation of the cuff pressure control portion according to the instruction given by the central process-and-control portion, by which compressed air is supplied to the cuff, and a predetermined pressure is exerted into the cuff. The speed and the degree of the pressure exerted into the cuff are applied by a program inputted in the central process-and-control portion. An example of the speed at which pressure is exerted into the cuff is about 50 mmHg/10 sec, the pressure exerted into the cuff being stepped up in several steps.

If the pressure into the cuff reaches a certain predetermined value (about 180 mmHg), the operation of the air supply portion stops by the instruction of the central process-and-control portion, starting the operation of the air exhaust portion, thereby the cuff pressure falling slowly. The speed at which the cuff pressure falls is applied according to the program provided.

In the rising and falling curves of the cuff pressure, the blood pressure measurement begins at the point where the korotkoff sound is detected by the pulse wave sensor (a kind of a microphone), i.e. at the point of about 90 mmHg of the rising curve of the cuff pressure; the maximal blood pressure is measured at its highest point (about 130 mm Hg); and the minimal blood pressure is measured at the point between about 100 mmHg and 80 mmHg of the falling curve.

At this time, in the present invention, as pulse waves are measured at the same time by the pulse wave sensor, the operation of the air exhaust portion stops at the point about 100 mmHg of the falling curve of the cuff pressure for one minute, thereby the same pressure being maintained horizontally. At this time, pulse waves are measured by the pulse wave sensor.

The foregoing is designed for precise measurement of pulse waves, a characteristic feature of the present invention, which is different from the conventional blood pressure measuring method. The reason for the one-minute stoppage of the cuff pressure is that the number of pulses is measured per minute as a unit.

Conventional pulse wave sensors (stethoscope microphones) are aimed mainly at the function of measuring the effective pressure range of blood pressure and the number of pulses. However, the main object of the pulse wave sensor in the present invention is different in that it comprises a compound function of measuring pulse waveforms, in addition to the said measuring of the effective pressure range and the number of pulses.

As conventional blood manometers are available with a pulse wave sensor, the function of the existing pulse wave sensor may be utilized in the embodiments of the present invention.

The data of the blood pressure that have been measured are amplified and filtered by an ordinary type blood pressure measuring portion and inputted into the central process-and-control portion. The central process-and-control portion then distinguishes the inputted blood pressure data between normal blood pressure and abnormal blood pressure. The abnormal blood pressure is further classified between hypertension and hypotension. Then the display portion displays the specific result of the measurement data in words, such as “normal blood pressure,” “hypertension,” and “hypotension.”

Meanwhile, the pulse wave data measured by the pulse wave sensor are inputted into the pulse wave measuring portion, and are inputted, through a process of amplifying and noise-filtering, into the A/D conversion portion that converts analog data into digital data. Then the digitalized pulse wave data are compared and analyzed by the comparison portion. The comparison portion then identifies the pulse wave data as one of the four types of pulse, i.e. normal pulse in which the peak cycles of the pulse waves per minute are steady, arrhythmia in which the peak cycles of the pulse waves are unsteady, tachycardia in which the peak cycles of the pulse waves are steady but the intervals between the peak cycles are abnormally short, and bradycardia in which abnormally long intervals are accompanied between the peak cycles of pulse waves. The data thus identified are inputted into the central process-and-control portion. The central process-and-control portion then determines the heart condition of the examined person, with the name of the disease identified as one of the six categories of the cardiac condition according to the inputted data, i.e. normal pulse, arrhythmia, tachycardia, bradycardia, hypertension, or hypotension. The storage portion stores the data of the cardiac condition with the name of a disease thus identified and determined, and the display portion displays the examined person's heart condition, showing the name of a cardiac disease in words, such as normal pulse, arrhythmia, tachycardia, bradycardia, hypertension, or hypotension, and where necessary, the data are outputted to a printer. The data stored in the central process-and-control portion may be transmitted to a remote-placed specialist recipient. by means of a mobile communication equipment.

### The Effect of the Invention

In the present invention, a combination of a blood pressure measuring means and a pulse wave measuring means is provided, making it possible to measure both blood pressure and pulse waves at the same time, to determine the heart condition of the examined person and to display the specific condition of the heart, showing the name of a cardiac disease identified. Therefore, different from the conventional simple blood manometer, a person may know her or his specific heart condition by measuring blood pressure. Even without a subjective symptom, the patient may read the name of his or her cardiac disease displayed, making it possible for the patient to consult with a remote-placed specialist through telemedicine and for more detailed and precise diagnosis without delay for proper treatment of a heart disease, thereby enhancing the effectiveness of the treatment.

The blood manometer in the present invention is small and portable, easy to use, convenient to send measured data through wireless transmission, whenever necessary, to a physician in charge who is located at a remote place, providing the user with a convenient means of physical health management.

### DESCRIPTION OF THE DRAWINGS

The above and other objects, features and other advantages of the present invention will be more clearly understood from the following detailed description taken in conjunction with the accompanying drawings, in which:

FIG. 1 shows the arrangement of the components of the blood manometer in the present invention.

FIG. 2 is a graph of the blood pressure measured in the present invention.

FIG. 3 illustrates the pulse waves measured in the present invention.

### EMBODIMENT

The preferred embodiments of the present invention will be explained in detail with reference to the accompanying drawings.

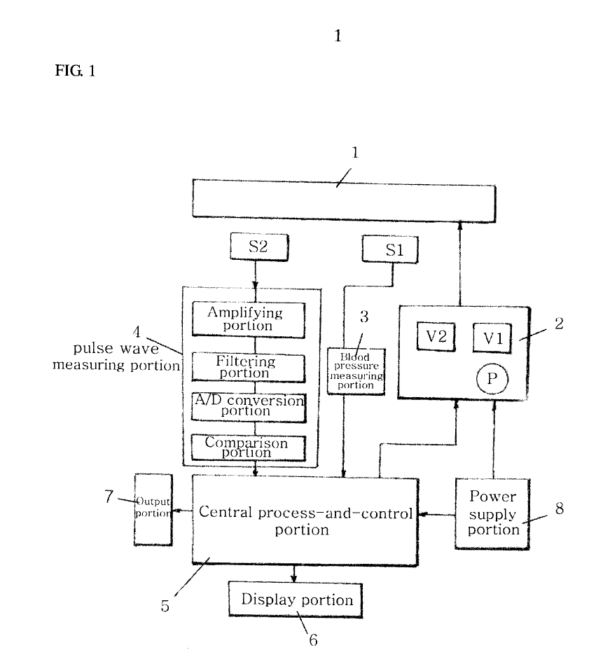

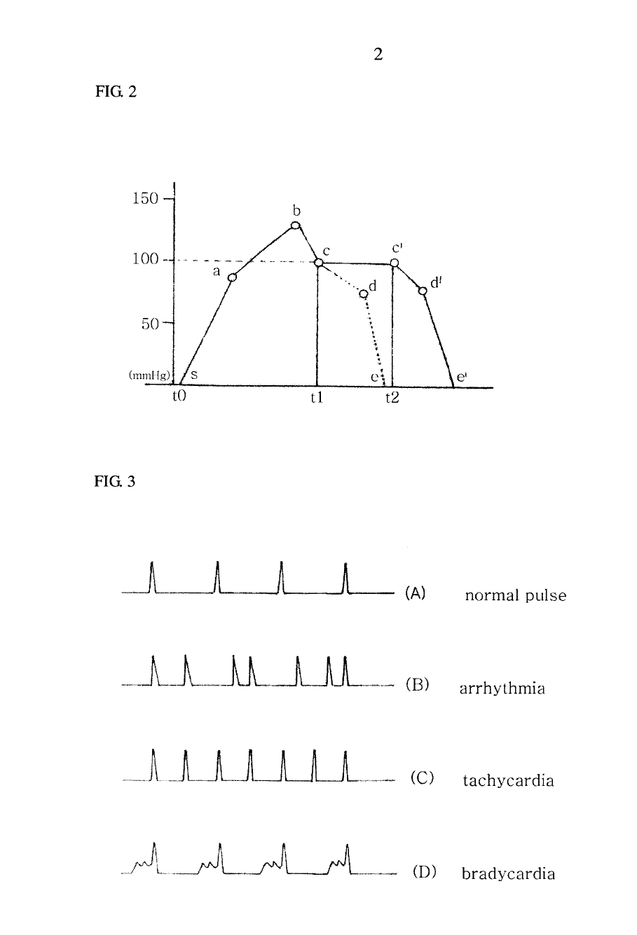

FIG. 1 shows the arrangement of the components of the blood manometer in the present invention; FIG. 2 is a graph of the blood pressure measured in the present invention; and FIG. 3 illustrates the pulse waves measured in the present invention.

The blood manometer in the present invention has a cuff (1) for measuring blood pressure, a cuff pressure control portion (2) for adjusting and controlling the air pressure in the cuff (1) and a blood pressure measuring portion (3) that comprises a pressure sensor (S1), and the blood manometer in the present invention is characterized in that it has a combined constitution of the blood pressure measuring portion (3) and a pulse wave measuring portion (4), which comprises a pulse wave sensor (S2) for measuring pulse waves, and a central process-and-control portion (5) for analyzing and processing the measured data outputted from both the blood pressure measuring portion (3) and the pulse wave measuring portion (4) and displaying in words the heart condition of the examined person, showing the name of a disease that is identified, through a display portion (6).

The pulse wave measuring portion (4) has a pulse wave sensor (S2) (stethoscope microphone), and comprises an amplifying portion for amplifying the pulse wave data of the pulse sound measured by the pulse wave sensor (S2), a filter portion for filtering external noises, an A/D conversion portion for converting the analogue waveform data into digital data, and a comparison portion for comparing and analyzing the form of the pulse waves and determining it as one of the predetermined pulse wave models, whereupon the data thus determined are inputted into the central process-and-control portion.

The central process-and-control portion (5) displays through the display portion (6) one of the three categories of the heart condition of the examined person, including the name of a cardiac disease identified, such as normal blood pressure, hypertension, and hypotension, according to the blood pressure measurement data inputted from the blood pressure measuring portion (3).

The central process-and-control portion (5) also displays through the display portion (6) the heart condition of the examined person according to the data of the pulse waveform measured by the pulse wave measuring portion (4), such as normal pulse, arrhythmia, tachycardia, or bradycardia.

An embodiment of the present invention is explained in detail as follows by the drawings attached.

As shown in FIG. 1, the blood manometer in the present invention has a cuff (1) for measuring blood pressure, a cuff pressure control portion (2) for adjusting and controlling the air pressure in the cuff (1) and a blood pressure measuring portion (3) that comprises a pressure sensor (S1), characterized in that the blood manometer has a combined constitution of a blood pressure measuring portion (3) and a pulse wave measuring portion (4), which comprises a pulse wave sensor (S2) for measuring pulse waves, and a central process-and-control portion (5) for analyzing and processing the measured data outputted from both the blood pressure measuring portion (3) and the pulse wave measuring portion (4) and displaying the heart condition of the examined person, showing the name of a disease identified, through a display portion (6) in words, the data thus displayed being stored and outputted to an output portion (7).

The cuff pressure control portion (2) comprises a pump (P) that provides compressed air in an ordinary manner, an air supply portion (V1) (an electronic valve) that injects air into the cuff (1) with a predetermined pressure, and an air exhaust portion (V2) that exhausts air from the cuff.

The blood pressure measuring portion (3), which is of an ordinary type, comprises a pressure sensor (S1), an amplifying portion for amplifying the measured blood pressure data and a filter portion for filtering noises (not shown in the drawings). The measured blood pressure data that have been processed are inputted into the central process-and-control portion (5).

The pulse wave measuring portion (4) has a pulse wave sensor (S2) (stethoscope microphone), and comprises an amplifying portion for amplifying the pulse waveform data of the pulse sound measured by the pulse wave sensor (S2), a filter portion for filtering external noises, an A/D conversion portion for converting analogue waveform data into digital data, and a comparison portion for comparing and analyzing the pulse waveform and determining it as one of the predetermined pulse wave models, whereupon the data thus determined are inputted into the central process-and-control portion (5).

The central process-and-control portion (5) consists of a kind of computer circuits and has a program inputted therein for controlling the operations of the cuff pressure control portion (2), the blood pressure measuring portion (3) and the pulse wave measuring portion (4). The central process-and-control portion (5) analyzes the measured data inputted from both the blood pressure measuring portion (3) and the pulse wave measuring portion (4), determines the heart condition of the examined person, showing the name of a cardiac disease identified, stores the analyzed data in the storage portion, and outputs the data to the display portion (6) and to the output portion (7). With a mobile communication equipment, such as a mobile phone, connected to the output portion (7), a wireless communication to a remote-placed physician for telemedicine is possible. The power supply portion (8) adjusts and supplies power with the voltage necessary for the operation of each component of the blood manometer.

The blood pressure measuring method of the present invention as stated above is carried out in an ordinary way, in which both the measuring of blood pressure and the measuring of pulse waves are done at the same time. In other words, the cuff (1) is wound around the designated portion of the body of the person examined. With an operation switch (not shown in the drawings) turned on, the power supply portion (8) supplies power to all the components of the blood manometer, activating all the components. According to the instruction given by the central process-and-control portion (5), a pump (P) is driven by the operation of the cuff pressure control portion (2), leading the air supply portion (V1) to supply compressed air into the cuff (1), applying predetermined pressure into the cuff (1). The speed at which pressure is exerted and the degree of the pressure are applied according to the program inputted in the central process-and-control portion (5). Under ordinary circumstances, the speed at which pressure is exerted into the cuff (1) is about 50 mmHg/10 sec. The speed of the pressure applied into the cuff may be variably adjusted.

If the cuff pressure reaches the predetermined level of 180 mmHg, the operation of the supply portion (V1) stops according to the instruction given by the central process-and-control portion (5), and the operation of the air exhaust portion (V2) begins, lowering the pressure in the cuff (1) slowly. The speed of the falling pressure is applied according to a predetermined program. The speed of the falling pressure is adjustable. An example of the falling speed is about 50 mmHg/12 sec.

The pressure curve at this time is shown in FIG. 2. Korotkoff sound is measured by the pulse wave sensor (S2) at the a point of the rising curve of the cuff pressure, i.e. near 90 mmHg. At this point where this measurement signal is detected, measuring of blood pressure starts by the pressure sensor (S1), and the maximal blood pressure is measured at the highest b point of the rising curve, i.e. at 130 mmHg. The minimal blood pressure is usually measured between the c point, i.e. 100 mmHg and the d point, i.e. 80 mmHg of the varying falling curve of the cuff pressure.

At this time, as pulse waves are measured at the same time by the pulse wave sensor (S2) in the present invention, in order to measure pulse waves accurately, the operation of the air exhaust portion (V2) stops for one minute at the c point of the varying cuff pressure falling curve, i.e. near 100 mmHg according to an instruction given by the central process-and-control portion (5), thereby maintaining the same pressure horizontally from the c point to the c′ point of the falling curve. During this period (t1-t2), pulse waves are measured by the pulse wave sensor (S2). At the d′ point, the measuring of the minimal blood pressure ends, and the cuff pressure begins to fall. Therefore, although the cuff pressure falling point of the varying cuff pressure curve is the e point shown in dotted line in the drawing, the variation of the cuff pressure stops for the period from t1 to t2 at the c point shown in full line. Therefore, the falling point of the varying cuff pressure curve is e′ point with the extension of the t1-t2 period. As such, the variation of the cuff pressure curve in measuring blood pressure in the present invention is characterized in that it contains a horizontal extended c-c′ line, different from that of a general type. This makes it possible to measure pulse waves precisely. The reason for the one-minute stoppage of the variation of the cuff pressure is that the number of pulses is measured by one minute as a unit. As the stoppage is applied automatically by the instruction of the central process-and-control portion, the examined person need not do anything to operate the device.

The data of the blood pressure within the effective pressure range of the blood pressure measurement measured by the pressure sensor, which is measured by the pulse wave sensor, is amplified, with an external noise-filtering process, by the ordinary blood pressure measuring portion (3) and inputted into the central process-and-control portion (5). The data of the measured blood pressure that have been inputted in the central process-and-control portion (5) are analyzed, identified and classified between normal blood pressure and abnormal blood pressure by an embedded computing program. In the case of abnormal blood pressure, it is further classified into hypertension and hypotension. The data thus identified and classified are stored in the storage portion. At the same time, the identified and classified data are displayed in words by the display portion (6) as “normal blood pressure,” “hypertension,” or “hypotension.”

Meanwhile, the pulse data that have been detected by the pulse sensor (S2) are inputted into the pulse measuring portion (4), amplified by the amplifying portion, and, with the process of filtering external noises by the filtering portion, converted from analog data into digital data by the A/D conversion portion. Then the pulse wave data are compared and analyzed by the comparison portion. As shown in FIG. 3, the pulse wave data thus compared and analyzed are classified into one of the four types of (A) in which the number of the peak cycles of the pulse waves per one minute is 60-70 and steady, (B) in which the peak cycles of pulse waves are irregular and unsteady, (C) in which the number of the peak cycles of pulse waves is over 80, which means the peak cycles are narrow, and (D) in which a prolonged pulse wave is accompanied between the peak cycles of pulse waves. The data thus identified and classified are inputted into the central process-and-control portion (5).

Then the central process-and-control portion determines the heart condition of the examined person, with the name of a cardiac disease identified, based on the inputted data, such as normal pulse for model A, arrhythmia for model B, tachycardia for model C, and bradycardia for model D. The data thus determined are stored in the storage portion of the central process-and-control portion (5), and at the same time the data are displayed in words on the display portion (6) like “normal pulse,” “arrhythmia,” “tachycardia,” “bradycardia.” When necessary, the data are printed at the output portion (7). By connecting a suitable mobile communication equipment to the output portion (7), the data stored in the central process-and-control portion may be transmitted via radio waves to a remote-placed specialist for telemedicine.

As the result of the measured blood pressure is displayed in words, the person, for whom the name of a cardiac disease is identified, may visit a physician without delay for further diagnosis and treatment.

After having used the blood manometer, the user may press the reset switch (not shown in the drawings) to stop the operation of all components before using it next time.

### INDUSTRIAL APPLICABILITY

The present invention is characterized in that measuring of blood pressure is followed at once by the display of the specific information on the cardiac disease. Therefore, this benefits an ordinary person at home for his or her health management. If an abnormal condition is displayed, the user may transmit the data to a physician at a remote place by means of a wireless mobile communication equipment, such as a mobile phone, for telemedicine and for necessary treatment without delay. A precise diagnosis from a specialist without delay enhances the chances of a better treatment of a heart disease.

1. A blood manometer displaying the name of a disease provided with a cuff (1) for measuring blood pressure, a cuff pressure control portion (2) for adjusting and controlling the air pressure in the cuff (1) and a blood pressure measuring portion (3) that comprises a pressure sensor (S1), characterized in that the blood manometer has a combined constitution of a blood pressure measuring portion (3) and a pulse wave measuring portion (4), which comprises a pulse wave sensor (S2) for measuring pulse waves, and a central process-and-control portion (5) for analyzing and processing the measured data outputted from both the blood pressure measuring portion (3) and the pulse wave measuring portion (4) and displaying in words the heart condition of the examined person, showing the name of a disease identified, through a display portion (6).

2. A blood manometer displaying the name of a disease as claimed in claim 1, in which the pulse wave measuring portion (4) has a pulse wave sensor (S2), and comprises an amplifying portion for amplifying the pulse waveform data of the pulse sound measured by the pulse wave sensor (S2), a filter portion for filtering external noises, an A/D conversion portion for converting analog waveform data into digital data, and a comparison portion for comparing and analyzing the pulse waveform and determining it as one of the predetermined pulse wave models, whereupon the data thus determined are inputted into the central process-and-control portion (5).

3. A blood manometer displaying the name of a disease as claimed in claim 1, in which the central process-and-control portion (5) displays through a display portion (6) the examined person's heart condition, showing the name of a disease identified, in words, such as normal blood pressure, hypertension, or hypotension, according to the blood pressure data measured and inputted by the blood pressure measuring portion (3),

4. A blood manometer displaying the name of a disease as claimed in claim 1, in which the central process-and-control portion (5) displays through a display portion (6) the examined person's heart condition, showing the name of a disease identified, such as normal pulse, arrhythmia, tachycardia, or bradycardia, according to the pulse waveform data models measured and outputted by the pulse wave measuring portion (4).
